(19) United States
(12) Patent Application Publication
(10) Pub. No.: US 2004/0054241 A1
(43) Pub. Date: Mar. 18, 2004

(54) Modified method for producing higher alpha-olefin

(76) Inventors: Heiko Maas, Mannheim (DE); Dag Wiebelhaus, Neustadt (DE); Jurgen Stephan, Mannheim (DE); Rocco Paciello, Bad Durkheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/466,544

(22) PCT Filed: Jan. 23, 2002
(86) PCT No.: PCT/EP02/00646

(30) Foreign Application Priority Data
Jan. 25, 2001 (DE) 10103309.5
Jun. 1, 2001 (DE) 10128048.3

(51) Int. Cl. C07C 2/00; C07C 4/00

(52) U.S. Cl. 585/324; 585/643

(57) ABSTRACT

A process for the targeted preparation of linear α-olefins having from 6 to 20 carbon comprises:

a) reaction of a linear, internal olefin or a mixture of linear, internal olefins having (n/2)+1 carbon atoms, where n is the number of carbon atoms in the desired linear α-olefin, with a trialkylaluminum compound in a transalkylation and isomerizing conditions, with an olefin corresponding to the alkyl radical being liberated and the linear olefin used adding onto the aluminum with isomerization and formation of a corresponding linear alkylaluminum compound,

b) reaction of the linear alkylaluminum compound formed with an olefin to liberate the corresponding linear α-olefin having (n/2)+1 carbon atoms and form a trialkylaluminum compound,

c) disproportionation of the linear α-olefin formed in a self-metathesis reaction to form a linear, internal olefin having the desired number n of carbon atoms,

d) reaction of the olefin having n carbon atoms which is formed with a trialkylaluminum compound under isomerization conditions, with an olefin corresponding to the alkyl radical being liberated and the linear, internal olefin adding onto the aluminum with isomerization and formation of a corresponding linear alkylaluminum compound,

e) reaction of the linear alkylaluminum compound formed with an olefin to liberate the linear α-olefin having the desired number n of carbon atoms and form a trialkylaluminum compound, and

f) isolation of the desired linear α-olefin having n carbon atoms.

The present invention relates to a process for preparing higher α-olefins by a combination of isomerizing transalkylation reactions with metathesis reactions.

Higher α-olefins have a lesser industrial importance than the short-chain olefins ethylene and propylene. There are nevertheless specific uses for each of the olefins belonging to this class, but there have hitherto been only general methods for preparing these higher olefins. Targeted syntheses are not possible. Thus, for example, the dehydrogenation of higher paraffins leads to a mixture of olefins which mostly contain internal double bonds. Olefins having a relatively high number of carbon atoms and terminal double bonds can be prepared by oligomerization of ethylene using transition metal catalysts, for example by the Ziegler process, the SHOP process of Shell or the Ethyl Process. However, the mixtures obtained have to be separated by sometimes very complicated methods if a particular α-olefin is to be isolated. In addition, ethylene is a high-priced starting material, since it is a raw material for a large number of chemical products. This naturally results in a higher price for the α-olefins obtained therefrom by oligomerization.

The higher α-olefins having 6 or more carbon atoms are gaining increasing importance, for example as comonomers in polyolefins. 1-Hexene and 1-octene are being used to an increasing extent in LLDPE (linear low density polyethylene). 1-Decene, for example, is gaining increasing importance as a starting material for the production of synthetic lubricants. There is therefore a great need for processes by means of which relatively long-chain α-olefins can be prepared in a targeted manner from starting materials other than ethylene.

According to EP-A 440 995, 1-octene can be prepared in a targeted manner from butadiene by telomerization and subsequent pyrolysis of the C8 telomerization product. Disadvantages of this process are the low yields and, in particular, the problem of catalyst recycling.

The metathesis of butene-containing streams is known, but only for the synthesis of olefins having up to 6 carbon atoms. For example, DE-A 100 13 253.7 describes the conversion of a mixture of 1-butene and 2-butene (raffinate II) into propene and 3-hexene, but formation of carbon chains longer than C6 cannot be achieved in this way.

U.S. Pat. No. 5,057,639 discloses a process for preparing 1-hexene, which comprises the process steps:

a) metathesis of 1-butene to form a mixture of 3-hexene and ethene;

b) separation of the 3-hexene from the product mixture obtained in step a);

c) reaction of the 3-hexene with an electrophile containing reactive hydrogen and preferably derived from water or a carboxylic acid under acid conditions which allows the addition of the electrophilic component onto the olefinic double bond;

d) cracking of the product from step c), for example by dehydration, to produce a mixture of n-hexenes in which 1-hexene is present in economically acceptable amounts.

This process does not make it possible for 1-hexene to be obtained selectively, since the cracking process leads only to a mixture of hexene isomers.

EP-A 505 834 and EP-A 525 760 both disclose a process for preparing linear higher α-olefins by successive transalkylation reactions. Here, a linear, internal olefin having from 4 to 30 carbon atoms or a mixture of such olefins is reacted with trialkylaluminum in the presence of an isomerization catalyst. This results in formation of a trialkylaluminum compound in which at least one of the alkyl radicals is derived from the olefin used; this radical is present as a linear alkyl radical derived from the α-olefin which has been formed by isomerization. The trialkylaluminum compound is subsequently reacted with an α-olefin in a displacement reaction in which the linear α-olefin which was bound to the aluminum is liberated.

This process allows internal olefins to be isomerized effectively and in good yields to produce terminal olefins. However, the process is a pure isomerization reaction which does not make it possible to increase the chain length. The internal olefins used for the isomerization come from the usual sources, and a targeted synthesis of α-olefins having a desired chain length is not possible by means of the process.

It is an object of the present invention to provide a process for the targeted preparation of particular relatively long-chain α-olefins. The process should, in particular, make it possible to use feedstocks other than the frequently employed, high-price lower olefins ethylene and propylene.

We have found that this object is achieved by a process for the targeted preparation of linear α-olefins having from 6 to 20 carbon atoms from linear internal olefins having a lower number of carbon atoms, which comprises the following steps:

a) reaction of a linear, internal olefin or a mixture of linear, internal olefins having (n/2)+1 carbon atoms, where n is the number of carbon atoms in the desired linear α-olefin, with a trialkylaluminum compound in a transalkylation under isomerizing conditions, with an olefin corresponding to the alkyl radical being liberated and the linear olefin used adding onto the aluminum with isomerization and formation of a corresponding linear alkylaluminum compound,

b) reaction of the linear alkylaluminum compound formed with an olefin to liberate the corresponding linear α-olefin having (n/2)+1 carbon atoms and form a trialkylaluminum compound,

c) disproportionation of the linear α-olefin formed in a self-metathesis reaction to form ethylene and a linear, internal olefin having the desired number n of carbon atoms,

d) reaction of the olefin having n carbon atoms which is formed with a trialkylaluminum compound under isomerization conditions, with an olefin corresponding to the alkyl radical being liberated and the linear, internal olefin adding onto the aluminum with isomerization and formation of a corresponding linear alkylaluminum compound,

e) reaction of the linear alkylaluminum compound formed with an olefin to liberate the linear α-olefin having the desired number n of carbon atoms and form a trialkylaluminum compound, and

f) isolation of the desired linear α-olefin having n carbon atoms.

For the purposes of the present invention, transalkylation is the reaction of an internal olefin with a trialkylaluminum compound under isomerizing conditions. The internal olefin undergoes rearrangement with double bond isomerization to give a mixture of internal and terminal olefins, and only the terminal olefins react to form a linear aluminum alkyl. An olefin which corresponds to the alkyl radical which was previously bound to the aluminum is then liberated.

In a preferred embodiment of the present invention, the olefin which is liberated in the reaction of the trialkylaluminum compound with the linear, internal olefin is isolated and reacted again with the trialkylaluminum compound formed.

In a further preferred embodiment of the present invention, the linear, internal olefins having (n/2)+1 carbon atoms and the linear, internal olefins having n carbon atoms are reacted jointly with the trialkylaluminum compound. In other words, the steps a) and d) are carried out together in one reaction space. The subsequent liberation of the α-olefins having (n/2)+1 and n carbon atoms (steps b) and e)) also occurs jointly. The mixture of linear α-olefins having (n/2)+1 carbon atoms and n carbon atoms liberated by reaction with an olefin is then fractionated, the olefin having (n/2)+1 carbon atoms is subjected to the self-metathesis reaction and the olefin having n carbon atoms is isolated.

Of course, it is also possible to use a mixture of linear internal olefins with linear terminal olefins as starting material. However, since the corresponding terminal olefins are frequently valuable chemical feedstocks, they are frequently removed from the mixture which is subsequently used in the process of the present invention.

In a variant of the present invention, a terminal olefin can also be used as starting material. In this case, the transalkylation a), i.e. the isomerization of the internal starting olefin to form a terminal olefin, becomes superfluous. The first step of the process of the invention is then the self-metathesis reaction of the olefin having (n/2)+1 carbon atoms, i.e. process step c). The subsequent process steps d) to f) are carried out in an unchanged manner.

A preferred product which can be prepared by the process of the present invention is 1-decene. In this case, the starting olefin used is a linear hexene or a mixture of various linear hexenes which is subjected to a transalkylation. This gives, after liberation, 1-hexene which is converted into 5-decene in a self-metathesis reaction. The latter olefin forms 1-decene in a further transalkylation.

Any hexene can be used in the reaction. In the above-described variant of the process of the present invention, in which the starting olefin used is a terminal olefin having (n/2)+1 carbon atoms, 1-hexene is used as starting olefin in the preparation of 1-decene. The latter is then subjected to a self-metathesis reaction to form 5-decene from which 1-decene is subsequently obtained.

In a preferred embodiment of the present invention, the hexene is obtained by metathesis of 1-butene, which forms 3-hexene. Possible sources of 1-butene are olefin mixtures which comprise 1-butene and 2-butene and possibly isobutene together with butanes. These are obtained, for example, in various cracking processes such as steam cracking or fluid catalytic cracking as C4 fraction. As an alternative, it is possible to use butene mixtures as are obtained in the dehydrogenation of butanes or by dimerization of ethene. Butanes present in the C4 fraction behave as inerts. Dienes, alkynes or enynes present in the mixture used are removed by means of customary methods such as extraction or selective hydrogenation.

The butene content of the C4 fraction used in the process is from 1 to 100% by weight, preferably from 60 to 90% by weight. Here, the butene content is the total content of 1-butene, 2-butene and isobutene.

Since olefin-containing C4-hydrocarbon mixtures are available at a favorable price, the use of these mixtures improves the addition of value to steam cracker by-products. Furthermore, products with high added value are obtained.

Preference is given to using a C4 fraction obtained in steam cracking or fluid catalytic cracking or in the dehydrogenation of butane.

The C4 fraction is particularly preferably used in the form of raffinate II, with the C4 stream being freed of interfering impurities, in particular oxygen compounds, by appropriate treatment over adsorber guard beds, preferably over high surface area aluminum oxides and/or molecular sieves. Raffinate II is obtained from the C4 fraction by firstly extracting butadiene and/or subjecting the stream to a selective hydrogenation. Removal of isobutene then gives the raffmate II.

Since the abovementioned mixtures comprise not only 1-butene but also internal olefins, the latter have to be converted into the terminal olefin prior to the metathesis reaction. This is achieved by a transalkylation in which the olefin mixture is reacted under isomerizing conditions with a trialkylaluminum compound. The 1-butene is subsequently liberated from the aluminum alkyl obtained by reaction with an olefin. The olefin liberated in the transalkylation of butene is preferably used, after isolation, for liberating the 1-butene.

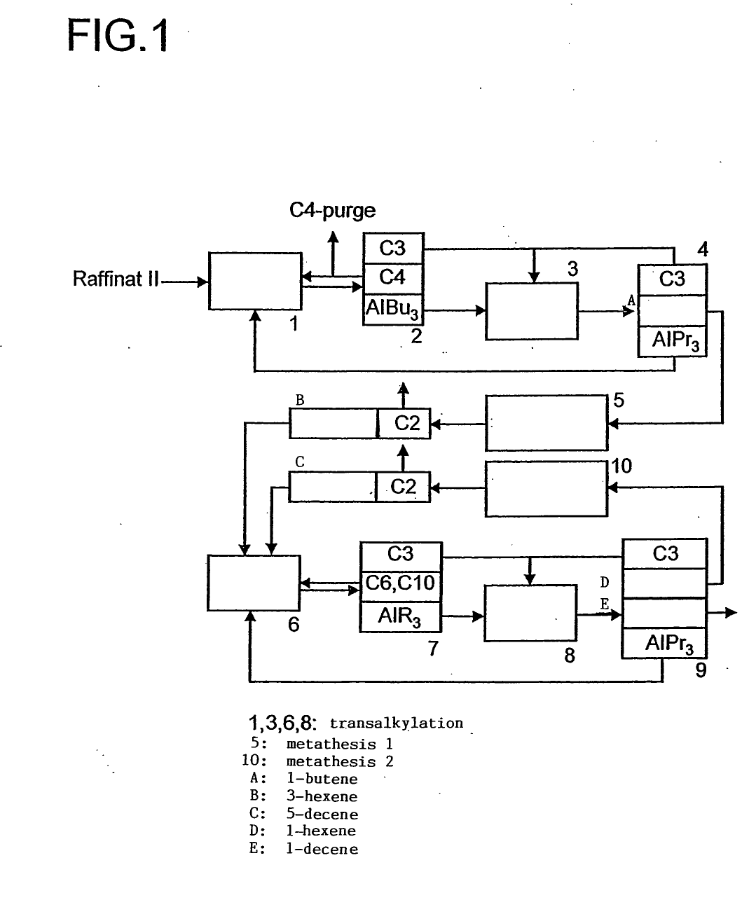

A preferred process for preparing 1-decene from raffmate II will now be described with reference to FIG. 1. Here, tripropylaluminum is used in each case as aluminum alkyl (see accompanying FIG. 1).

In a first transalkylation (1), raffinate II is reacted with tripropylaluminum to form tri-n-butylaluminum and propene. Propene and the excess of C4 fraction are separated off (2), and the C4 is returned to the transalkylation. In the subsequent transalkylation (3), the tri-n-butylaluminum is reacted with the previously isolated propene to form tripropylaluminum and 1-butene. Excess propene is isolated and recirculated. The tripropylaluminum obtained is used in the transalkylation (1). The 1-butene is subjected to a self-metathesis reaction to form 3-hexene and ethylene (5). The valuable product ethylene is separated off and utilized elsewhere. The 3-hexene formed is then subjected to a transalkylation using tripropylaluminum (6), with 5-decene, which is a downstream product (see below), also being fed into the reactor. Mixed C3-/C6-/C10-alkyls of aluminum are formed. In reaction step (7), the excesses of 3-hexene and 5-decene are separated off and recirculated, while the mixed aluminum alkyls formed are reacted with propene in reaction step (8) to form tripropylaluminum and a mixture of 1-hexene and 1-decene. Excess propene is recirculated. Tripropylaluminum is used again in the transalkylation step (6). 1-Decene is discharged as product (9). In this variant of the process, 1-hexene is used in a self-metathesis reaction (10) to produce 5-decene. The ethylene formed in this reaction is discharged as product of value and is utilized elsewhere. The 5-decene obtained is passed to the transalkylation (6).

The above-described process has the advantage that not only 1-decene but also ethylene are formed as product of value.

The self-metathesis of 1-butene to form 3-hexene and ethene is known in principle and is described in Chem. Tech. 1986, page 112, and in U.S. Pat. No. 3,448,163. The self-metathesis of 1-hexene to form ethene and 5-decene is likewise known and is described, for example, in J. Jpn. Petrol. Inst. 1983, 26, page 332, and in Rec. Trav. Chim. Pays Bas 1977, 96, M 31. All these processes use isomerically pure α-olefins which are prepared exclusively by oligomerization of ethylene and only internal olefins are obtainable by this self-metathesis.

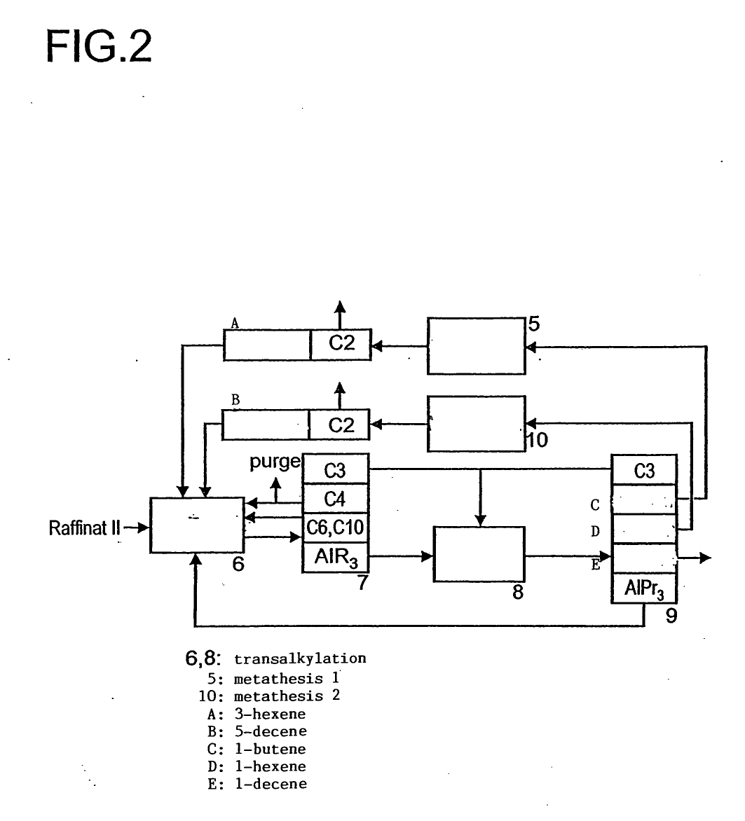

In a further, preferred variant of the process of the present invention, butenes together with 3-hexene and 5-decene are jointly used as starting material for the transalkylation. This is shown in FIG. 2 in which the reference numerals have the meanings defined in FIG. 1 (see accompanying FIG. 2).

The mixture of trialkylaluminum, butene, hexene and decene as well as propene obtained after the transalkylation reaction (6) with tripropylaluminum is fractionated (7). The C4-, C6- and C10-olefins are returned to the reaction, propene and aluminum alkyl are passed to a further transalkylation (8) in which 1-butene, 1-hexene and 1-decene are formed (9).

These are separated, and 1-decene is isolated and 1-butene and 1-hexene are subjected to a self-metathesis reaction (5 and 10). The C3 stream is circulated. The products 3-hexene and 5-decene leaving the metathesis reactor are used in the transalkylation (6). Ethylene formed is separated off and utilized elsewhere.

In a further, preferred embodiment of the process of the present invention, the 3-hexene is obtained from a C4 olefin mixture, in particular raffmate II, by carrying out a metathesis reaction as described in DE 100 13 253.7 (Applicant: BASF AG). This reaction comprises the following steps:

a) The raffmate II starting stream, which preferably has a high 1-butene content as a result of appropriate choice of the parameters in the preceding selective hydrogenation of butadiene, is subjected, optionally with addition of ethene, to a metathesis reaction in the presence of a metathesis catalyst comprising at least one compound of a metal of group VIb, VIIb or VIII of the Periodic Table of the Elements to convert the butenes present in the starting stream into a mixture comprising ethene, propene, butenes, 2-pentene, 3-hexene and butanes, with ethene, if employed, being used in an amount of from 0.05 to 0.6 molar equivalents based on the butenes.

b) The starting stream obtained in this way is firstly subjected to fractional distillation to give a low-boiling fraction A comprising C2-C3-olefins and a high-boiling fraction comprising C4-C6-olefins and butanes.

c) The low-boiling fraction A obtained from b) is subsequently fractionally distilled to give an ethene-containing fraction and a propene-containing fraction, with the ethene-containing fraction being recirculated to the process step a) and the propene-containing fraction being discharged as product.

d) The high-boiling fraction obtained from b) is subsequently fractionally distilled to give a low-boiling fraction B comprising butenes and butanes, a middle fraction C comprising pentene and a high-boiling fraction D comprising hexene.

e) The fractions B and C are recirculated in full or in part to the process step a), and the fraction D is discharged as product.

3-Hexene and propene are obtained in various ratios in this reaction.

The raffinate II starting stream is obtained from the C4 fraction by customary methods known to those skilled in the art, with interfering isobutene and butadiene being removed. Suitable processes are disclosed in the patent application DE 100 13 253.7.

Depending on the respective demand for the products propene and 3-hexene, the external mass balance of the process can be influenced in a targeted way by variable input of ethene and by recirculation of particular substreams to shift the equilibrium. Thus, for example, the yield of 3-hexene is increased by recirculation of 2-pentene to the metathesis step in order to suppress the cross-metathesis of 1-butene with 2-butene, so that little if any 1-butene is consumed here.

The self-metathesis of 1-butene to form 3-hexene which then proceeds preferentially additionally forms ethylene which reacts with 2-butene in a subsequent reaction to form the valuable product propene.

The metathesis process of DE 100 13 253.7 is an integral part of the present invention and is incorporated by reference.

After the propene has been separated off, the 3-hexene is then subjected to a transalkylation using aluminum alkyls. Otherwise, the process is carried out in the same way as when hexene is obtained from raffinate II by transalkylation and subsequent metathesis.

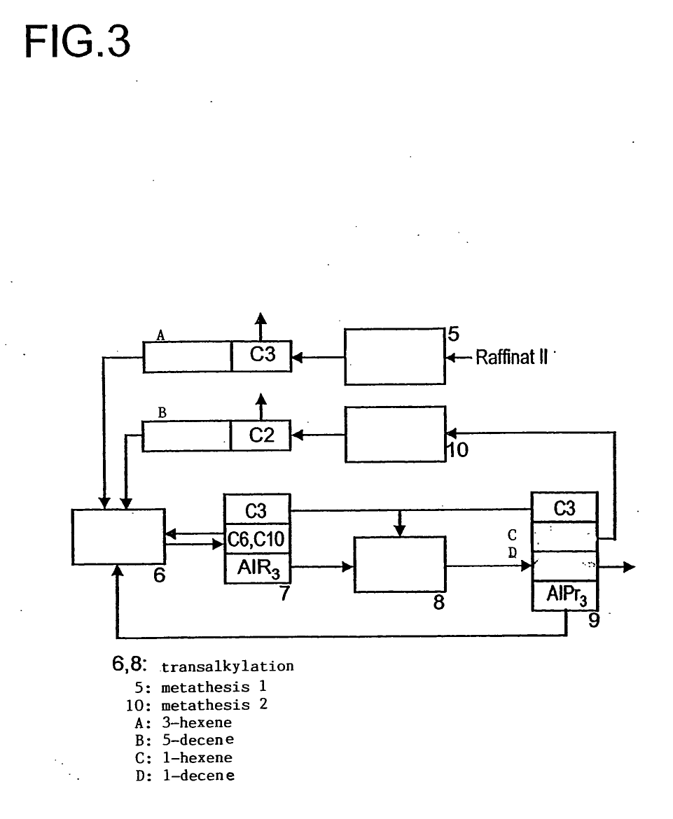

In the present embodiment, too, a preferred embodiment comprises carrying out the transalkylation of the olefin having (n/2)+1 carbon atoms and the olefin having n carbon atoms jointly in one reactor. This preferred embodiment is shown in FIG. 3. Here, (5) denotes the reactor in which the process as described in DE 100 13 253.7 is carried out. The remaining reference numerals have the meanings defined in FIG. 1 (see accompanying FIG. 3).

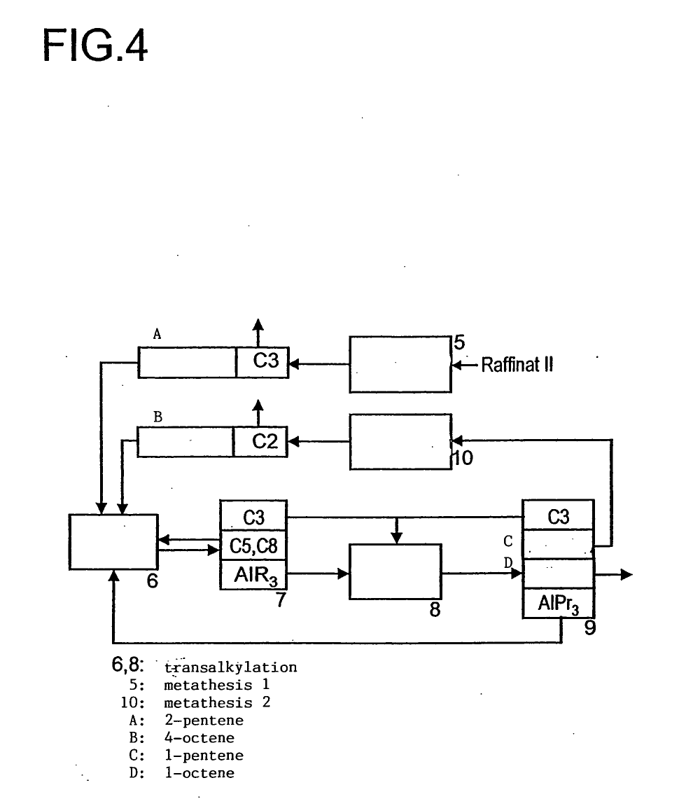

A further preferred product which can be prepared by means of the process of the present invention is 1-octene, which is used to an increasing extent as comonomer in LLDPE. Here, linear pentene or a mixture of various linear pentenes is used as starting material. This process will be described with reference to FIG. 4 below, which shows a preferred embodiment. In the process shown in FIG. 4, the transalkylation of 2-pentene and that of 2-octene are carried out jointly, which is preferred according to the present invention. However, the transalkylation reactions for each of these two olefins can be carried out separately (see accompanying FIG. 4).

The starting olefin used is linear, internal pentene, preferably 2-pentene. This is subjected to a transalkylation (6) using tripropylaluminum, with 4-octene, which is a downstream product (see below), also being fed into the reactor. Mixed C3-/C5-/C8-alkyls of aluminum are formed. In reaction step (7), the excesses of 2-pentene and 4-octene are separated off and recirculated, while the mixed aluminum alkyls formed are reacted with propene in reaction step (8) to form tripropylaluminum and a mixture of 1-pentene and 1-octene. Excess propene is recirculated. Tripropylaluminum is used again in the transalkylation step (6). 1-Octene is discharged as product (9). In this variant of the process, 1-pentene is used for producing 4-octene in a self-metathesis reaction (10). The ethylene formed in this reaction is discharged as valuable product and is utilized elsewhere. The 4-octene obtained is used in the transalkylation (6).

The above-described process has, in particular, the advantage that not only 1-octene but also ethylene are formed as product of value.

It is of course also possible here to use the terminal olefin, i.e. 1-pentene, as starting material, in which case steps a) and b) according to the invention are dispensed with.

In a preferred variant of the present invention, a C4-containing olefin stream, in particular raffinate II is used for preparing pentene. The starting olefin mixture is then converted into 2-pentene and propene using the process described in DE 199 32 060.8, as shown in FIG. 4. The process comprises the following steps:

a) The raffinate II starting stream, which has a suitable ratio of 1-butene to 2-butene as a result of appropriate choice of the parameters in the preceding selective hydrogenation of butadiene, is subjected, optionally with addition of ethene, to a metathesis reaction in the presence of a metathesis catalyst comprising at least one compound of a metal of group VIb, VIIb or VIII of the Periodic Table of the Elements to convert the butenes present in the starting stream into a mixture comprising ethene, propene, butenes, 2-pentene, 3-hexene and butanes, with ethene, if employed, being used in an amount of from 0.05 to 0.6 molar equivalents based on the butenes.

b) The starting stream obtained in this way is firstly subjected to fractional distillation to give a low-boiling fraction A comprising C2-C3-olefins and a high-boiling fraction comprising C4-C6-olefins and butanes.

c) The low-boiling fraction A obtained from b) is subsequently fractionally distilled to give an ethene-containing fraction and a propene-containing fraction, with the ethene-containing fraction being recirculated to the process step a) and the propene-containing fraction being discharged as product.

d) The high-boiling fraction obtained from b) is subsequently fractionally distilled to give a low-boiling fraction B comprising butenes and butanes, a middle fraction C comprising pentene and a high-boiling fraction D comprising hexene.

e) The fractions B and D are recirculated in full or in part to the process step a), and the fraction C is discharged as product.

2-Pentene and propene are obtained in various ratios in this reaction.

The raffinate II starting stream used preferably has a high 2-butene content, at least a 2-butene/1-butene ratio of 1.

The raffinate II starting stream is obtained from the C4 fraction by customary methods known to those skilled in the art, with interfering isobutene and butadiene being removed. Suitable processes are disclosed in the patent application DE 199 32 060.8.

Depending on the respective demand for the products propene and 2-pentene, the external mass balance of the process can be influenced in a targeted way by variable input of ethene and by recirculation of particular substreams to shift the equilibrium. Thus, for example, the 2-pentene yield can be increased by recirculating or of the C4 fraction obtained in step d) and all of the C5 fraction obtained in step d) to the metathesis reaction.

The metathesis reaction described in DE 199 32 060.8 is an integral part of the present invention and is incorporated by reference.

In all variants of the process of the present invention, the olefin liberated in the transalkylation is preferably removed continuously from the reactor.

The catalysts used in the self-metathesis comprise a compound of a metal of group VIb, VIIb or VIII of the Periodic Table of the Elements. The catalysts can be applied to inorganic supports. The metathesis catalyst preferably comprises an oxide of a metal of group VIb or VIIb of the Periodic Table of the Elements. In particular, the metathesis catalyst is selected from the group consisting of Re₂O₇, WO₃ and MoO₃. The most preferred catalyst is Re₂O₇ applied to y-Al₂O₃ or mixed Al₂O₃/B₂O₃/SiO₂ supports.

The metathesis reaction can be carried out either in the gas phase or in the liquid phase. The temperatures are from 0 to 200° C., preferably from 40 to 150° C., and the pressures are from 20 to 80 bar, preferably from 30 to 50 bar.

In the transalkylation reaction a linear, internal olefin having from 4 to 30 carbon atoms or a mixture of such olefins having internal double bonds is reacted with a trialkylaluminum compound in a molar ratio of the linear olefins having internal double bonds to trialkylaluminum of from 1 to a maximum of 50/1. The reaction is carried out in the presence of a catalytic amount of a nickel-containing isomerization catalyst which effects the isomerization of the internal olefinic double bond, as a result of which at least a small amount of linear α-olefin is produced. The alkyl groups are subsequently displaced from the trialkylaluminum to form a new alkylaluminum compound in which at least one of the alkyl groups bound to the aluminum is a linear alkyl derived from the corresponding linear α-olefin. The alkylaluminum compound is subsequently reacted with a 1-olefin in the presence of a displacement catalyst in order to displace the linear alkyl from the alkylaluminum compound and produce a free, linear α-olefin. The isomerization catalyst is selected from among nickel(II) salts, nickel(II) carboxylates, nickel(II) acetonates and nickel(0) complexes, which may be stabilized by means of a trivalent phosphorus ligand. In another embodiment, the isomerization catalyst is selected from the group consisting of bis-1,5-cyclooctadienenickel, nickel acetate, nickel naphthenate, nickel octanoate, nickel 2-ethylhexanoate and nickel chloride.

An appropriate transalkylation processes is described in the patent applications EP-A 505 834 and EP-A 525 760. The context of these applications is an integral part of the present invention and is incorporated by reference.

The transalkylation reaction can also be carried out using variants which are known to or can be deduced by a person skilled in the art. In particular, it is possible to use isomerization catalysts which contain no Ni or no Ni compound.

The aluminum alkyls used in the transalkylation are known to those skilled in the art. They are selected according to availability or, for example, aspects relating to the way the reaction is carried out. Examples of such compounds include triethylaluminum, tripropylaluminum, tri-n-butylaluminum and triisobutylaluminum. Preference is given to using tripropylaluminum or triethylaluminum.

The invention will now be laid out in the following examples

### EXAMPLE 1

Metathesis of Raffinate II into 3-hexene

General Process:

Raffinate II of the respective composition, fresh ethene and the respective C4- and C5-recycle stream are mixed, in the respective ratio, thereafter the metathesis reaction is carried out in a 500 ml tube reactor using a 10% Re₂O₇-catalyst. The discharge is then separated into a C2/3-, C4-, CS- and a C6-stream using three columns, thereafter every stream is analyzed by GC. The C4-stream is then split up and divided into a C4-purge and a C4-recycle.

The balances given below were recorded over 24 h at constant reaction temperature.

### EXAMPLE 1.A

| | raffinate | | C4- | | C5- | dis- | dis- |
|---|---|---|---|---|---|---|---|
| | II | | re- | C4- | re- | charge | charge |
| | fresh | ethene | cycle | purge | cycle | 3-hexene | propene |
| stream | 660 g/h | 100 | 1470 | 190 | 440 | 190 g/h | 320 g/h |
| | | g/h | g/h | g/h | g/h | | |

Composition raffinate II:

| | |
|---|---|
| butanes¹⁾ | 90 g/h |
| 1-butene | 330 g/h |
| 2-butene²⁾ | 240 g/h |

¹⁾sum isobutane + n-butane
²⁾sum cis + trans

### EXAMPLE 1.B

| | raffinates | | C4- | | C5- | dis- | dis- |
|---|---|---|---|---|---|---|---|
| | II | ethene | re- | C4- | re- | charge | charge |
| | fresh | fresh | cycle | purge | cycle | 3-hexene | propene |
| stream | 500 g/h | 80 | 1120 | 160 | 380 | 110 g/h | 240 g/h |
| | | g/h | g/h | g/h | g/h | | |

composition raffinate II:

| | |
|---|---|
| butanes¹⁾ | 100 g/h |
| 1-butene | 200 g/h |
| 2-butene²⁾ | 200 g/h |

¹⁾sum isobutane + n-butane
²⁾sum cos + trans

### EXAMPLE 1.C

| | raffinate | | C4- | | C5- | dis- | dis- |
|---|---|---|---|---|---|---|---|
| | II | ethene | re- | C4- | re- | charge | charge |
| | fresh | fresh | cycle | purge | cycle | 3-hexene | propene |
| stream | 660 g/h | 100 | 1470 | 230 | 540 | 180 | 300 |
| | | g/h | g/h | g/h | g/h | | |

Composition raffinate II:

| | |
|---|---|
| butanes¹⁾ | 90 g/h |
| 1-butene | 330 g/h |
| 2-butene²⁾ | 240 g/h |

¹⁾sum isobutane + n-butane
²⁾sum cis + trans

### EXAMPLE 2

### Isomerization of 3-hexene into 1-hexene

2.1: Isomerizing Transalkylation

General Process:

3-Hexene and tripropylaluminum (hydride content ≦1000 ppm) are mixed in a molar ratio of 10:1. The mixture is heated to reflux, then a defined quantity of nickel salt in toluene is added, thereafter the propene formed is removed. The amount of trihexylaluminum is calculated by taking samples which are hydrolyzed with aqueous HCl and analyzing the organic phase by GC. The amount of n-hexane found corresponds to the amount of trihexylaluminum originally formed.

### EXAMPLE 2.1.A

100 ppm nickel in form of nickel acetylacetonate, added over 2 minutes;

yield trihexylaluminum from tripropylaluminum:

69.0% after 60 min.,

76.1% after 120 min.

### EXAMPLE 2.1.B

20 ppm nickel in form of nickel naphthenate, added over 5 min.;

yield of trihexylaluminum from tripropylaluminum:

21.8% after 45 min.

### EXAMPLE 2.1.C

200 ppm nickel in form of nickel acetylacetonate, added over 30 min.;

yield of trihexylaluminum from tripropylaluminum:

96.8% after 60 min.

2.2: Catalytic Displacement

General Process:

Trihexylaluminum is put into an autoclave, thereafter the autoclave is pressurized using the same mass of propene. The reaction is started by adding a defined amount of nickel salt in toluene, at room temperature. Samples are taken after certain times, which samples are hydrolyzed by aqueous HCl. The organic phase is analyzed by CG, the amounts of hexene formed are determined.

### EXAMPLE 2.2.A

20 ppm nickel in form of nickel naphthenate; conversion of aluminum trihexyl: 35% after 30 min.; α-olefin proportion of hexenes formed: 89%

### EXAMPLE 2.2.B

50 ppm nickel in form of nickel acetylacetonate; conversion of aluminum trihexyl: 50% after 10 min.; α-olefin proportion of hexenes formed: 96%

### EXAMPLE 3

### Metathesis of 1-hexene to 5-decene

The catalyst (10% Re₂O₇ on Al₂O₃) is given into a reaction vessel, under protective atmosphere, thereafter 1-hexene is added. The reaction starts spontaneously, a gas (ethene) develops vigorously. Stirring is continued at room temperature, after a defined time the liquid phase is analyzed by GC. Conversion is 80% after 24 h, the selectivity is 99%.

### EXAMPLE 4

### Isomerization of 5-decene to 1-decene

### EXAMPLE 4.A

### Isomerizing Transalkylation

5-Decene and tripropylaluminum (hydride content <1000 ppm) are mixed in a molar ratio of 10:1. The mixture is heated to reflux, then 100 ppm nickel in form of nickel acetylacetonate, in toluene, are added over 2 min., thereafter the propene formed is removed. The amount of tridecylaluminum formed is calculated by taking samples at various times, which samples are hydrolyzed by aqueous HCl, and analyzing the organic phase by GC. The quantity of n-decane found corresponds to the amount of tridecylaluminum originally formed.

Yield of tridecylaluminum formed from tripropylaluminum: 73.5% after 60 min.

### EXAMPLE 4.B

### Catalytic Displacement

Tridecylaluminum is given into an autoclave, which is pressurized using the same mass of propene. The reaction is started by adding 40 ppm nickel in form of nickel naphthenate in toluene, at room temperature. After various times, samples are taken which are hydrolyzed by aqueous HCl. The organic phase is analyzed by GC and the amount of decenes is determined.

Conversion of aluminum tridecyl: 50% after 15 min.;

α-olefin proportion of hexenes formed: 92%

We claim:

1. A process for the targeted preparation of linear α-olefins having from 6 to 20 carbon atoms from linear internal olefins having a lower number of carbon atoms, which comprises the following steps:
a) reaction of a linear, internal olefin or a mixture of linear, internal olefins having (n/2)+1 carbon atoms, where n is the number of carbon atoms in the desired linear α-olefin, with a trialkylaluminum compound in a transalkylation under isomerizing conditions, with an olefin corresponding to the alkyl radical being liberated and the linear olefin used adding onto the aluminum with isomerization and formation of a corresponding linear alkylaluminum compound,
b) reaction of the linear alkylaluminum compound formed with an olefin to liberate the corresponding linear α-olefin having (n/2)+1 carbon atoms and form a trialkylaluminum compound,
c) disproportionation of the linear α-olefin formed in a self-metathesis reaction to form ethylene and a linear, internal olefin having the desired number n of carbon atoms,
d) reaction of the olefin having n carbon atoms which is formed with a trialkylaluminum compound under isomerization conditions, with an olefin corresponding to the alkyl radical being liberated and the linear, internal olefin adding onto the aluminum with isomerization and formation of a corresponding linear alkylaluminum compound,
e) reaction of the linear alkylaluminum compound formed with an olefin to liberate the linear α-olefin having the desired number n of carbon atoms and form a trialkylaluminum compound, and
f) isolation of the desired linear α-olefin having n carbon atoms.

2. A process as claimed in claim 1, wherein the linear, internal olefins having (n/2)+1 carbon atoms and the linear, internal olefins having n carbon atoms are reacted jointly with the trialkylaluminum compound and the corresponding α-olefins are liberated jointly from the trialkylaluminum compounds formed.

3. A process as claimed in claim 1 or 2, wherein 1-decene is prepared using a hexene or a hexene mixture, preferably 3-hexene, in step a).

4. A process as claimed in claim 3, wherein 3-hexene is prepared by self-metathesis of 1-butene.

5. A process as claimed in claim 4, wherein 1-butene is prepared from butene-containing streams, preferably raffinate II by transalkylation and isomerizing conditions.

6. A process as claimed in any of claims 3 to 5, wherein the butene-containing streams, the linear, internal olefins having (n/2)+1 carbon atoms and the linear, internal olefins having n carbon atoms are reacted jointly with the trialkylaluminum compound and the corresponding α-olefins are liberated jointly from the trialkylaluminum compounds formed.

7. A process as claimed in claim 3, wherein hexene is prepared from butene-containing streams, preferably raffinate II, using the following steps:
a′ metathesis of the starting material, optionally with addition of ethene,
b′ fractional distillation of the stream obtained to give a low-boiling fraction A comprising C2-C3-olefins and a high-boiling fraction comprising C4-C6-olefins and butanes,
c′ fractional distillation of the low-boiling fraction obtained to give an ethene-containing fraction and a propene-containing fraction, with the ethene-containing fraction being recirculated to process step a′ and the propene-containing fraction being discharged as product,
d′ fractional distillation of the high-boiling fraction obtained to give a low-boiling fraction comprising butenes and butanes, a middle fraction comprising pentene and a high-boiling fraction comprising hexene, and
e′ discharge of the hexene-containing high-boiling fraction and optional recirculation of the other fractions to process step a′.

8. A process as claimed in claim 1 or 2, wherein steps a) and b) are omitted and a linear α-olefin, preferably 1-hexene, is subjected to the self-metathesis reaction c).

9. A process as claimed in claim 1 or 2, wherein 1-octene is prepared using a pentene or a pentene mixture, preferably 2-pentene, in step a).

10. A process as claimed in claim 9, wherein 2-pentene is prepared from butene-containing streams, preferably raffmate II, which preferably have a ratio of 2-butene to 1-butene of at least 1, preferably using the following steps:
a′ metathesis of the starting material, optionally with addition of ethene,
b′ fractional distillation of the stream obtained to give a low-boiling fraction A comprising C2-C3-olefins and a high-boiling fraction comprising C4-C6-olefins and butanes,
c′ fractional distillation of the low-boiling fraction obtained to give an ethene-containing fraction and a propene-containing fraction, with the ethene-containing fraction being recirculated to process step a′ and the propene-containing fraction being discharged as product,
d′ fractional distillation of the high-boiling fraction obtained to give a low-boiling fraction comprising butenes and butanes, a middle fraction comprising pentene and a high-boiling fraction comprising hexene, and
e′ discharge of the pentene-containing middle fraction and optional recirculation of the other fractions to process step a′.

11. A process as claimed in any of claims 1 to 10, wherein the olefin liberated in the transalkylation steps a) and/or d) is removed continuously from the reactor and/or is used for liberation of the α-olefins in steps b) and/or e).

12. A process as claimed in any of claims 1 to 11, wherein the catalyst used in the self-metathesis may have been applied to inorganic supports and comprises a compound of a metal of group VIb, VIIb or VIII of the Period Table of the Elements, preferably an oxide of a metal of group VIb or VIIb of the Periodic Table of the Elements, where the metathesis catalyst is particularly preferably selected from the group consisting of Re₂O₇, WO₃ and MoO₃ and is most preferably Re₂O₇ which has been applied to γ-Al₂O₃ or to mixed Al₂O₃/B₂O₃/SiO₂ supports.

13. A process as claimed in any of claims 1 to 12, wherein a homogeneous catalyst is employed.

14. A process as claimed in any of claims 1 to 13, wherein the self-metathesis reaction is carried out at from 0 to 200° C., preferably from 40 to 150° C., at pressures of from 20 to 80 bar, preferably from 30 to 50 bar.

15. A process as claimed in any of claims 1 to 14, wherein the aluminum alkyl used is a trialkylaluminum compound having C2-C10-alkyl radicals, preferably tripropylaluminum or triethylaluminum.
